(19) United States
(12) Patent Application Publication
(10) Pub. No.: US 2022/0387454 A1
(43) Pub. Date: Dec. 8, 2022

(54) NUTRITIONAL COMPOSITION FOR VISUAL FUNCTION

(71) Applicant: OMNIACTIVE HEALTH TECHNOLOGIES LIMITED, Mumbai, Maharashtra (IN)

(72) Inventors: Muralidhara PADIGARU, Thane (W), Maharashtra (IN); Abhijeet MORDE, Thane (W), Maharashtra (IN); Prakash BHANUSE, Thane (W), Maharashtra (IN)

(73) Assignee: OMNIACTIVE HEALTH TECHNOLOGIES LIMITED, Mumbai, Maharashtra (IN)

(21) Appl. No.: 17/762,727

(22) PCT Filed: Nov. 10, 2020
(86) PCT No.: PCT/IB2020/060572

§ 371 Date: Mar. 22, 2022

(30) Foreign Application Priority Data
Nov. 15, 2019 (IN) 201921046568

(51) Int. Cl. A61K 31/593; A61K 31/047; A61K 31/12; A61K 47/44; A61K 47/24; A61K 47/14; A61P 27/04

(52) U.S. Cl. CPC A61K 31/593; A61K 31/047; A61K 31/12; A61K 47/44; A61K 47/24; A61K 47/14; A61P 27/04

(57) ABSTRACT

A nutritional composition for prevention, improvement and maintenance of visual function. The nutritional composition comprising the nutrients and at least one pharmaceutically and/or nutraceutically accepted excipient. The nutritional composition wherein the nutrients are selected from one or more of lutein, curcumin and vitamin D3 for prevention, improvement and maintenance of dry eye. The process of preparation of the nutritional composition comprising lutein, curcumin and vitamin D3 use for visual function more specifically dry eye. The present invention is further encompassing to the nutritional composition comprising lutein, curcumin, vitamin D3 in selective percentage which is formulated in different forms like as, powders, granules, pellets, beadlets, caplets, tablets, capsules, soft gel capsules, solution, emulsions, suspensions, oil suspensions, dispersions and the like.

### FIELD OF THE PRESENT INVENTION

The present invention is related to the nutritional composition use for prevention, improvement and maintenance of visual function in a subject in need thereof. More particularly, the invention describes a nutritional composition comprising the nutrients and at least one pharmaceutically and/or nutraceutically accepted excipients. More specifically, the invention is related to the nutritional composition wherein the nutrients are selected from one or more of lutein, zeaxanthin, curcumin and vitamin D3 use for prevention, improvement and maintenance of visual function and in dry eye syndrome. Further to this invention is related to process of preparation of a nutritional composition comprising lutein, zeaxanthin, curcumin and vitamin D3 use for visual function more specifically for dry eye syndrome.

### BACKGROUND OF THE PRESENT INVENTION

Dry eye syndrome (DES) is a multifactorial disease of the tear and ocular surface that result in reduced tear production, symptoms of discomfort and visual disturbance with potential damage to the ocular surface. It is mainly associated with tear film instability, increased osmolarity of the tear and ocular surface inflammation. Oxidative stress plays an important role in inducing damages to the ocular surface in DES with increased levels of malondialdehyde (MDA), lipid peroxidation markers and decreased antioxidants such as superoxide dismutase (SOD), catalase, and glutathione peroxidase in the tear film and ocular surface of dry eye patients.

Lutein and zeaxanthin are present in the eye in high concentrations. The retina and lens in general and the macular region in the centre of the retina in particular are highly enriched in these two xanthophylls. Lutein and zeaxanthin absorb high energy blue light and protects retina from phototoxicity. Oxidative stress occurs as a result of the disruption of the balance between the antioxidant system and the pro-oxidant system found in cells. It has been accepted that overexpression of ROS can be induced in the ocular surface as a result of many acute and chronic diseases and even in normal aging. Recent studies demonstrated that oxidative stress damages the ocular surface and plays an important role in the mechanism of dry eye disease. Curcumin, a yellow coloured polyphenol from the plant Curcuma longa and known for anti-inflammatory and antioxidant properties was used another ingredient in the present formulation. Studies have demonstrated beneficial effects of curcumin in multiple anterior segment eye diseases such as corneal disease, dry eye condition, conjunctivitis, anterior uveitis, cataracts and glaucoma. Vitamin D is a multifunctional vitamin that is now known to exert significant roles in many biological activities.

More importantly, low serum vitamin D levels are found to be associated with DES, and tear secretion and tear breakup time (TBUT) were positively correlated with serum vitamin D concentrations. Furthermore, vitamin D receptor is found in the corneal epithelium, endothelium and retinal pigmentary epithelium and also enhances corneal epithelial barrier function. Vitamin D regulates fluid and ion transport in salivary glands and may regulate tear secretion in the lacrimal glands.

EP 338 478 0 A1 relates to a composition comprising linseed oil and hempseed oil having a balanced omega-3 and omega-6 ratio and/or a volume/volume ratio between linseed oil and hempseed oil comprised between 60:40 and 95:5 and its process of preparation. The composition further contains vitamins, polyphenols, flavonoids, iso-flavonoids, bioflavonoids, phytoestrogens, carotenoids, vegetable extracts, lactic ferments, melatonin, coenzyme Q10, lipophilic substances, molecules with a particularly rapid metabolism, and combinations thereof.

US20060020046 A1 is speaks about the use of lycopene, optionally in combination with vitamin E and/or C or other biologically active ingredients as disclosed in the Specification, in the manufacture of a composition for the primary and secondary prevention of angiogenesis-associated pathologies and coadjutant treatment thereof, as well as with particular novel formulations comprising lycopene.

WO2018235939A1 provides an ophthalmic composition containing a clathrate antioxidant substance.

WO2008113177A1 provide various compounds and compositions comprising polyunsaturated fatty acid monoglycerides and derivatives thereof. These compounds and compositions can be useful as cancer chemo preventive agents. They can also be useful for enhancing solubility of various active agents and enhancing their bioavailability.

The above all said references are completely silent over the composition comprising lutein, zeaxanthin, curcumin and vitamin D3 use for visual function and related complications such as dry eye syndrome. As the literatures speaks the first line of treatment for DES consists use of artificial tears followed by topical corticosteroids which usually improve the symptoms of the disease but are associated with adverse effects over long term use. Hence there has been a lot of interest in exploring the effectiveness of natural ingredients that can be administered orally avoiding frequent use and adverse events associated with long term use of eye drops. There is long felt need to provide a nutritional composition with selective nutrients use for prevention, improvement and maintenance visual functions more particularly dry eye syndrome including of a subject in need thereof.

### OBJECTS OF THE PRESENT INVENTION

The primary objective of the present invention is to develop a nutritional composition comprising the nutrients selected from one or more of lutein, zeaxanthin, curcumin and vitamin D3 and at least one pharmaceutically and/or nutraceutically accepted excipients for prevention, improvement and maintenance of visual function.

Another objective of the present invention is to develop a nutritional composition comprising lutein, zeaxanthin, curcumin and vitamin D3 use for prevention, improvement and maintenance of dry eye syndrome.

Further objective of the present invention is to provide a nutritional composition comprising lutein, zeaxanthin, curcumin and vitamin D3 in selective percentage and/or ratio which may formulated in different forms like orally administrable solid, semisolid, liquid forms, selected from, but not limited to dosages such as, powders, granules, pellets, beadlets, caplets, tablets, capsules, soft gel capsules, solution, emulsions, suspensions, oil suspensions, dispersions and the like.

Another objective of the present invention is to reduce inflammatory cytokines such as nuclear factor kappa light chain enhancer of activated B cells (NFkB), Matrix metallopeptidase 9 (MMP-9), tumor necrosis factor alpha (TNF-α), Interleukin 1 beta (IL-1b), Interleukin 6 (IL-6) and Interleukin 8 (IL-8).

One more objective of the present invention is to reduce oxidative stress marker such as Malondialdehyde (MDA) and increase antioxidant enzymes like superoxide dismutase (SOD) and glutathione peroxidase (GPx).

Further objective of the present invention is to increase tear volume, tear break-up time, tear stability, increased mucin production.

Another objective of the present invention is to reduce corneal surface damage, conjunctival surface damage, artificial tear usage and frequency.

One more objective of the present invention is to develop a composition of that significantly enhances tear production, soothes dry eyes, reduces eye discomfort, improves tear stability, reduces irritation, burning and grittiness associated with dry eyes, reduces of tear loss, reduces ocular surface damage, reduces inflammation in the tears and reduces dependence on artificial tears.

Further objective of the present invention is to provide a process of preparation of a nutritional composition comprising lutein, zeaxanthin, curcumin and vitamin D3.

### BRIEF DESCRIPTION OF ACCOMPANYING FIGURES

FIG. 1: Mean Schirmer's Test Strip Wetness Length in mm (Overall Subjects—Average of Both Eyes) Between Test Product (Active) and Placebo Groups.

FIG. 2: Mean Ocular Surface Disease Index (OSDI) Scores Between Test Product (Active) and Placebo Groups.

FIG. 3: Mean TBUT Score (Overall Subjects—Average of Both Eyes) Between Test Product (Active) and Placebo Groups.

FIG. 4: Mean Tear Osmolarity (Overall Subjects—Average of Both Eyes) Between Test Product (Active) and Placebo Groups.

FIG. 5: Mean Standardized Patient Evaluation of Eye Dryness (SPEED) Questionnaire Total Scores for Test Product (Active) and Placebo Groups

FIG. 6: Mean Corneal Staining Score (Overall Subjects—Average of Both Eyes) Between Test Product (Active) and Placebo Groups

FIG. 7: Mean Conjunctival Staining Score (Overall Subjects—Average of Both Eyes) Between Test Product (Active) and Placebo Groups

FIG. 8: Number of Subjects Positive for MMP-9 Biomarker (Overall Subjects—Right Eye) Between Test Product (Active) and Placebo Groups

FIG. 9: Number of Subjects Positive for MMP-9 Biomarker (Overall Subjects—Left Eye) Between Test Product (Active) and Placebo Groups.

FIG. 10: Number of Subjects Used Artificial Tears (Overall Subjects) Between Test Product (Active) and Placebo Groups.

FIG. 11: Mean Frequency of Artificial Tears Usage (Overall Subjects) Between Test Product (Active) and Placebo Groups.

FIG. 12. Effect of the combination of lutein, zeaxanthin curcumin and vitamin D (LCD) on serum malondialdehyde (MDA), corneal MDA, corneal superoxide dismutase (SOD), and corneal glutathione peroxidase (GSH-Px), in benzalkonium chloride (BAC) induced dry eye syndrome in rats.

FIG. 13. Effect of the combination of lutein, zeaxanthin, curcumin and vitamin D (LCD) on nuclear factor-kappa B (NF-κB), tumour necrosis factor alpha (TNF-α), interleukin 1 beta, interleukin 6 (IL-6), and interleukin 8 (IL-8) protein levels in benzalkonium chloride (BAC) induced dry eye syndrome in rats.

FIG. 14: The histological appearance of the corneas in rats. H&E ×400. Control: The cornea had a normal histological appearance.

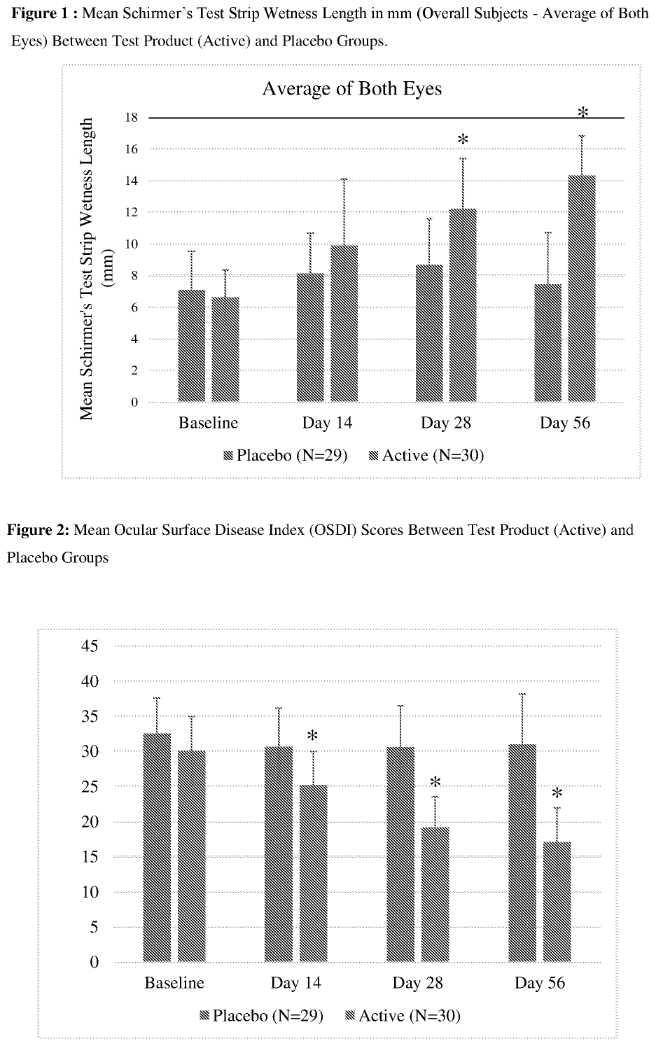

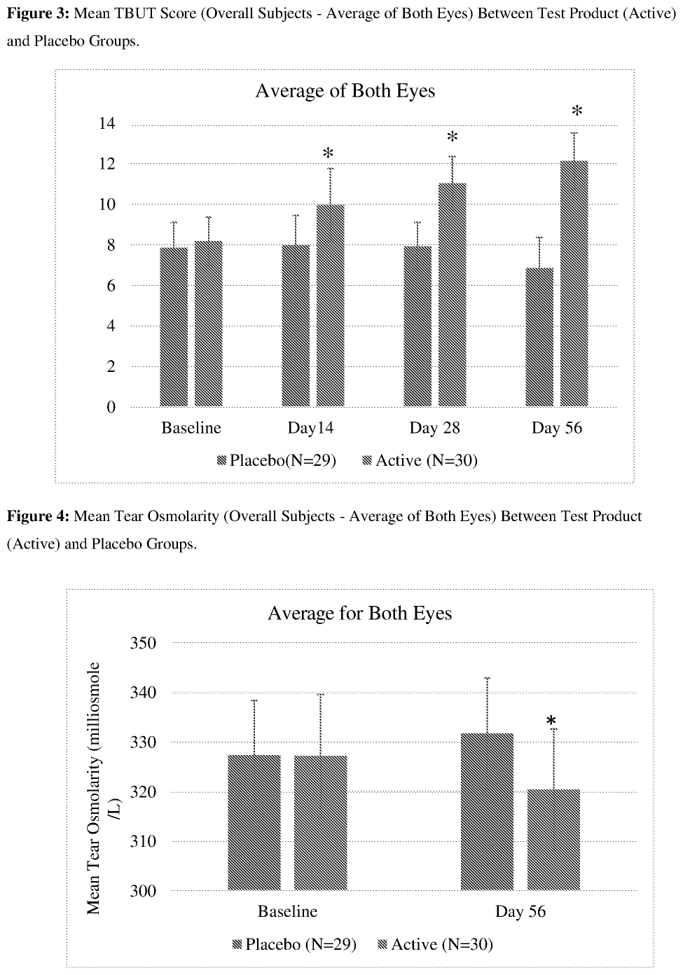

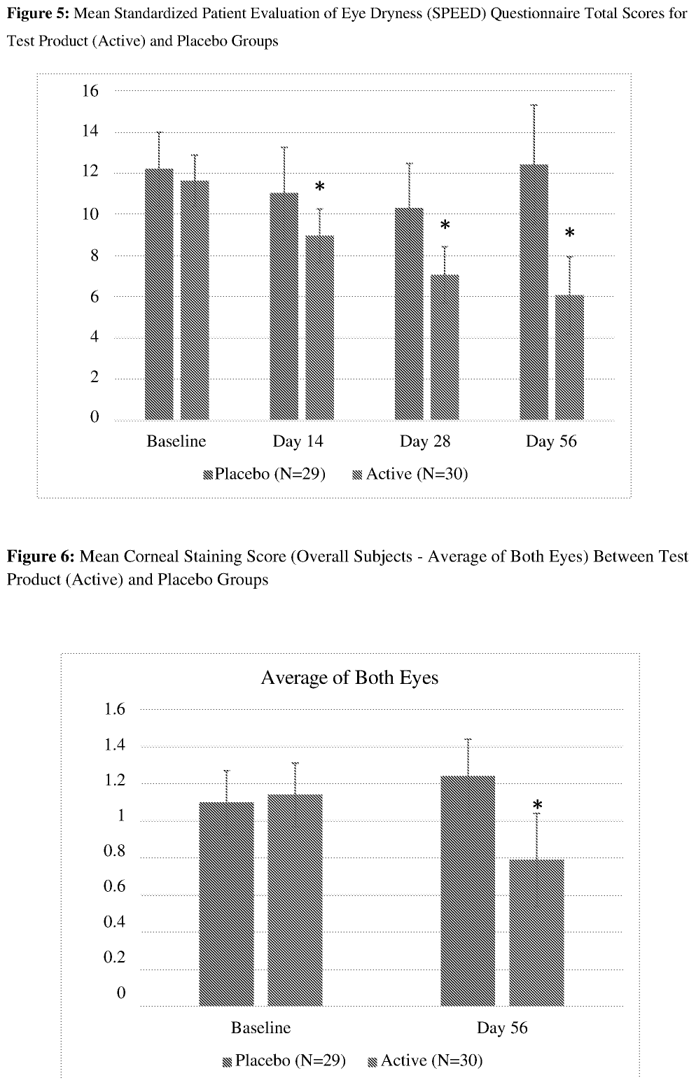

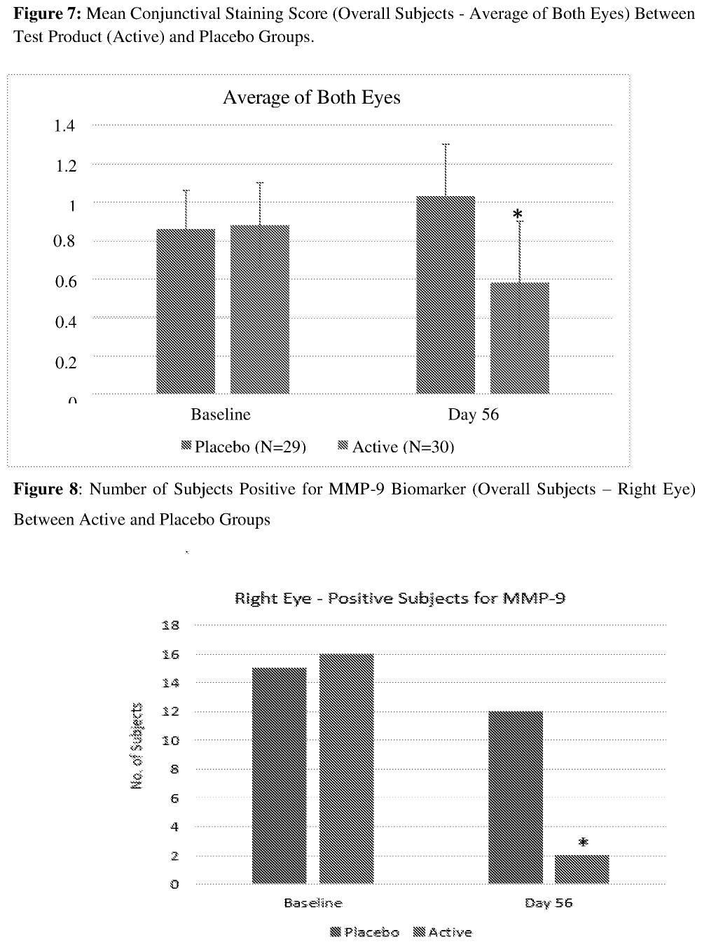

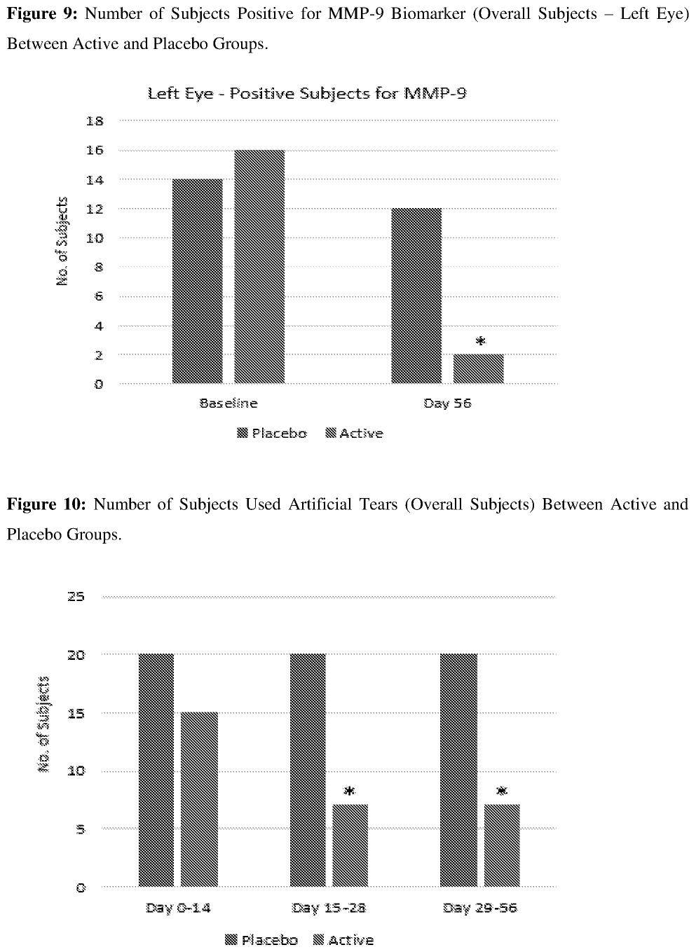

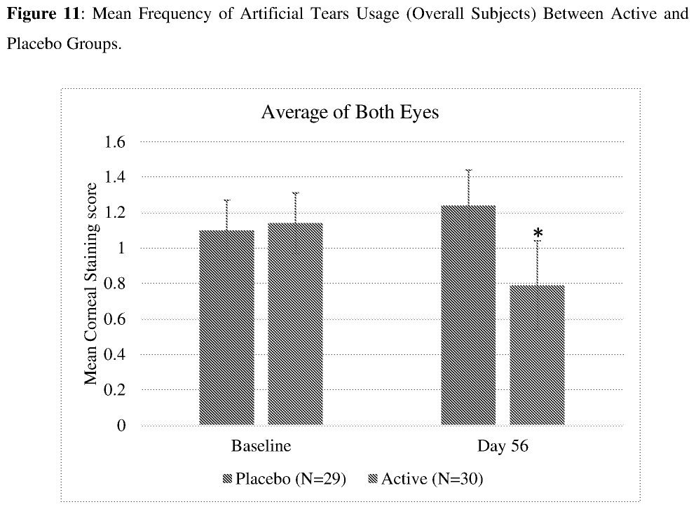

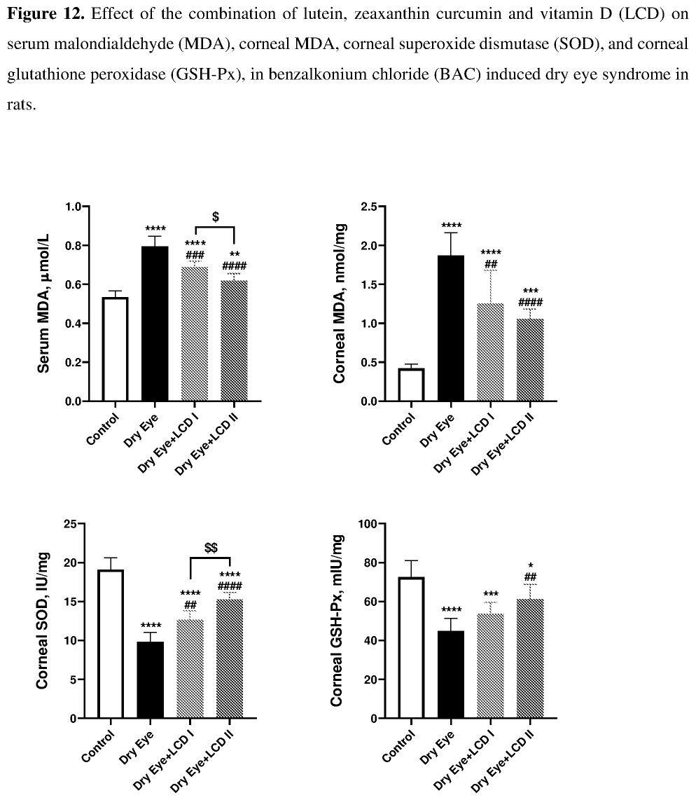

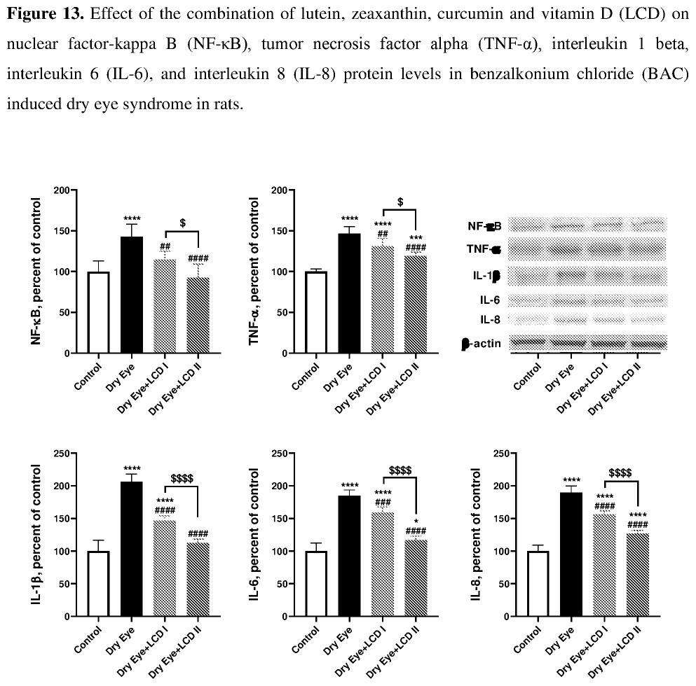

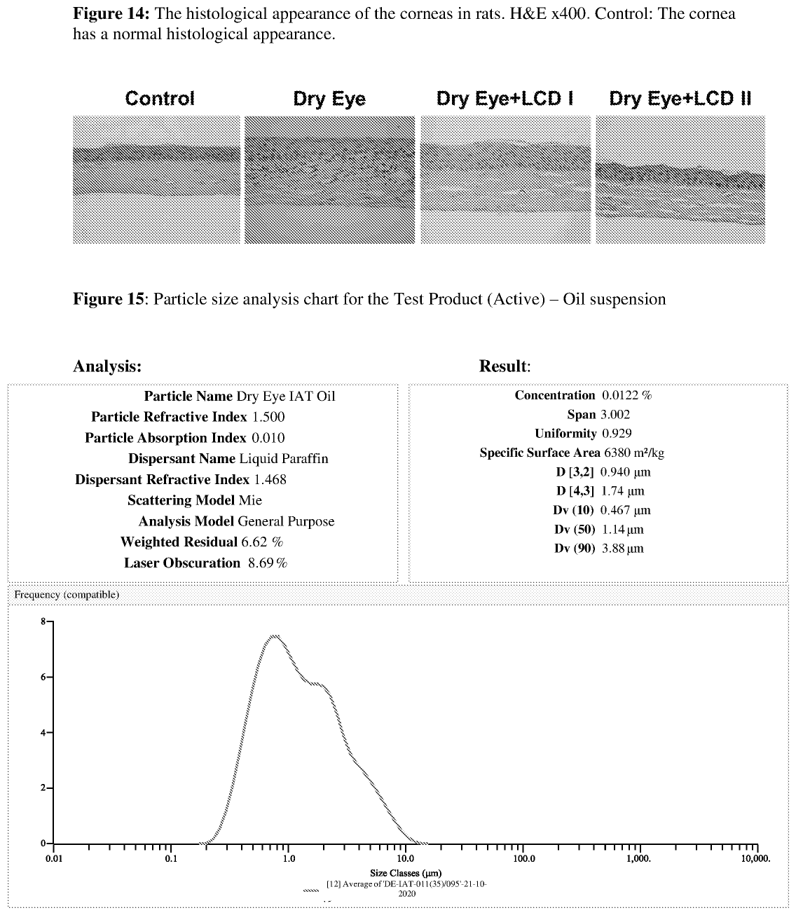

### DETAILED DESCRIPTION OF THE PRESENT INVENTION

The invention described herein relates to a nutritional composition and use for prevention, improvement and maintenance of visual functions more particularly to dry eye syndrome. Within the context of this invention the terminology the “a nutritional composition” is commonly used in the specification to refer composition having selectively added the nutrients such as lutein, zeaxanthin, curcumin and Vitamin D3, with one or more of pharmaceutically and/or nutraceutically accepted excipients which promote enhancement in result.

The inventors of this invention have arrived on this selective composition of active ingredients by rigorous experimentations wherein none of the arts speaks about the composition with the use for dry eye syndrome. we came across with below documents which speaks about the single ingredient may work for dry eye syndrome with inadequate experimentation without any motivation for comparison.

In published article https://www.eyeworld.org/article-vitamin-d-and-dry-eye-is-there-an-association speaks about vitamin D3 induces production of IL-10, which inhibits production of other pro-inflammatory cytokines, including IL-1, IL-6 and TNF-alpha. So considering this It makes physiological sense that vitamin D3 would also play a role in assisting with the inflammatory mediators in the tear film in patients suffering with dry eye.

As per the published article in nature.com with title “Vitamin D Supplementation for Patients with Dry Eye Syndrome Refractory to Conventional Treatment” Scientific Reports 6:33083 DOI: 10.1038/srep33083 speaks about the study investigated the effect of vitamin D supplementation in patients with dry eye syndrome (DES) refractory to conventional treatment with vitamin D deficiency. A total of 105 patients with DES refractory to conventional treatment and vitamin D deficiency that was treated with an intramuscular injection of cholecalciferol (200,000 IU). Serum 25-hydroxyvitamin D (25(OH)D) levels were measured. Eye discomfort was assessed using ocular surface disease index (OSDI) and visual analogue pain score (VAS). Tear break-up time (TBUT), fluorescein staining score (FSS), eyelid margin hyperemia, and tear secretion test were measured before treatment, and 2, 6, and 10 weeks after vitamin D supplementation. Mean serum 25(OH)D level was 10.52±4.61 ng/mL. TBUT, and tear secretion test showed an improvement at 2 and 6 weeks after vitamin D supplementation compared to pre-treatment values (p<0.05 for all, paired t-test). Eyelid margin hyperemia and the severity of symptoms showed improvement at 2, 6, and 10 weeks after vitamin D supplementation (p<0.05 for all). Compared to pre-treatment values, FSS, OSDI and VAS were decreased at 2 weeks (p<0.05 for all). In conclusion, vitamin D supplementation is effective and useful in the treatment of patients with DES refractory to conventional treatment and with vitamin D deficiency.

However, both the published article is talking about vit D3 only and completely silent over the composition of lutein, zeaxanthin, curcumin and Vit D3. So, considering the comparison of this instant invention against the article reflects significant improvement over due to selective composition with formulation.

In published article Pescosolido N et al. Curcumin: Therapeutically Potential Planta Medicine 2014; 80: 249-254, It has been demonstrated that curcumin has beneficial effects on several ocular diseases, such as chronic anterior uveitis, diabetic retinopathy, glaucoma, age-related macular degeneration, and dry eye syndrome. The purpose of this review is to report what has so far been elucidated about curcumin properties and its potential use in ophthalmology.

By careful inspection of the prior arts available in public domain and referred above, all the arts are completely silent over on the claimed composition of lutein, zeaxanthin, curcumin and vitamin D3. However, some of the arts are with singly ingredient merely speaks about dry eye syndrome with inadequate experimentation support. But none of the prior art teaches suggest or motivate about the composition of this instant invention.

The embodiments of this instant invention are well elaborated as follows:

According to the embodiment of the present invention, the nutrients are selected from one or more lutein, zeaxanthin, curcumin, vitamin D3, ginger, ashwagandha and betacryptoxanthin and/or mixture thereof.

As per one more embodiment of the present invention, a nutritional composition comprising the nutrients and one or more of pharmaceutically and/or nutraceutically accepted excipients use for prevention, improvement and maintenance of dry eye syndrome.

According to important embodiment of the present invention, a nutritional composition comprising the nutrients selected from lutein, zeaxanthin, curcumin, and vitamin D3 with one or more of pharmaceutically and/or nutraceutically accepted excipients use for prevention, improvement and maintenance of dry eye syndrome.

According to one more embodiment of the present invention a nutritional compositions of the invention are comprised of the nutrients such as curcumin [1,7%-bis(4-hydroxy-3-methoxyphenyl)-1,6-heptadien-3,5-dione] which is a polyphenol derivative derived from the spice turmeric. Curcumin is a major active ingredient of Curcuma Longa. Curcuma longa (turmeric) is a well-known indigenous herbal medicine. It is to be appreciated that the term “curcumin” can be interpreted to be within the scope of the term curcuminoids, which can in general include components curcumin, methoxy curcumin, demethoxy curcumin, bisdemethoxy curcumin and tetrahydrocurcumin.

According to further embodiment the curcumin is present in range of 20-50% by weigh of composition.

According to embodiment of the present invention a nutritional composition of the invention is comprised of the nutrients such as lutein an extract from the plant material selected for preparation of the composition is marigold flower (Tagetes eracta). Particularly lutein ester rich extract is obtained from marigold flower. The marigold extract prepared in-house by saponification and thermal isomerization reaction of marigold flower oleoresin. The final product contains at least 80% of the total carotenoids with 60 to 85% lutein and 10-20% zeaxanthin isomers. In an embodiment, the marigold flower extract is comprised of lutein and zeaxanthin isomers in the ratio of 4:1 to 6:1.

According to further embodiment the lutein and zeaxanthin is present in range of 2-10% by weigh of composition.

According to further embodiment vitamin D3 is present in range of 0.01-2% by weigh of composition.

A nutritional composition described herein are comprised of lutein, zeaxanthin, curcumin and vitamin D3 either alone or in combination with at least one or more pharmaceutically and/or nutraceutically acceptable excipients increases solubility and hence enhance an absorption of composition. More preferably, nutritional composition is formulated using excipients selected from the group of, but not limited to antioxidants, stabilizers, carriers, fats, solubilizes and bioavailability enhancers or the combination thereof.

In one more embodiment, the carrier employed in the preparation of a nutritional compositions is selected from the group such as, but not limited to milk fat, medium chain triglycerides (MCT), long chain triglycerides, oils such as flaxseed oil, Olive Oil, thyme oil, fish oil, algal DHA Oil, krill Oil, safflower Oil, sunflower oil, soybean oil, coconut oil, other vegetable oils, esters of fatty acids, hydrocarbons such as terpenes, monoglycerides and derivative thereof.

According to preferred embodiment the carrier used is medium chain triglycerides (MCT) oil. According to further embodiment MCT oil carrier present in range of 10-60% by weight of composition. According to further embodiment MCT oil carrier present in range of 20-50% by weight of composition.

In one more embodiment, antioxidant employed in preparation of nutritional compositions is selected from the group such as, but not limited to, natural mixed tocopherols, ascorbyl palmitate, rosemary extract, epigallocatechin gallate, catechins, ascorbic acid and derivative thereof.

According to preferred embodiment antioxidant is a natural mixed tocopherol.

According to further embodiment, antioxidant present in range of 1-10% by weight of composition. According to further embodiment the antioxidant present in range of 1-5% by weight of composition. According to further embodiment the antioxidant present in range of 1-3% by weight of composition.

In one more embodiment, bioavailability enhancers employed in preparation of nutritional compositions is selected from the group such as, but not limited to, olive oil, thyme oil, linseed oil, d-limonene, monoglycerides, phospholipids such as lecithin/phosphatidyl choline, botanical extracts and derivative thereof. The bioavailability enhancer also acts as a solubility enhancer.

According to further embodiment, bioavailability enhancing agent present in range of 1-20% by weigh of composition. According to further embodiment the bioavailability enhancing agent present in range of 1-10% by weigh of composition. According to further embodiment the bioavailability enhancing agent present in range of 1-5% by weigh of composition.

In one more embodiment, the stabilizer employed in preparation of a nutritional compositions is selected from the group such as, but not limited to sugar alcohols, triglycerides, antioxidants and derivative thereof.

In one more embodiment, fat employed in preparation of a nutritional compositions is selected from the group such as, but not limited to, milk fat, esters of fatty acids, hydrocarbons such as terpenes, monoglycerides and derivative thereof.

In one more embodiment, the solubilizer in preparation of a nutritional compositions selected from the group such as, but not limited to propylene glycol alginate, sugar alcohols, sugar esters, phospholipid, Vitamin E TPGS (d-α-Tocopheryl polyethylene glycol 1000 succinate), beta cyclodextrin and/or combination thereof.

In one more embodiment, the pH modifier in preparation of a nutritional compositions is selected from the group such as, but not limited to citric acid, trisodium citrate, lactic acid, L-arginine, Calcium carbonate, magnesium carbonate, and/or combination thereof.

A nutritional composition described herein exhibit in enhancement of bioavailability and the compositions can be available in orally administrable solid, semisolid, liquid forms, selected from, but not limited to dosages such as, oil suspensions, powders, granules, pellets, beadlets, caplets, tablets, capsules, soft gel capsules, solution, emulsions, suspensions, dispersions and the like.

As per the preferred embodiment, a nutritional composition is in the form of oil suspension comprising nutrients and pharmaceutically and/or nutraceutically acceptable excipient.

According to further embodiment of the present invention, the obtained oil suspension composition having particle size is in the range of 0.1-10 micron. The particle size of the active herbal ingredients before micronization and after micronization has observed and provided in table no 01. The composition particle size observed that micronization has a significant impact on composition (oil suspension) of lutein, zeaxanthin, curcumin and vitamin D3, reduced the particle size approximately ten times for all the components measured in our experiments.

Particle Size Determination of Particles.

Particle size analysis is performed by using Malvern instrument with the details provided below:

| | PSD(D90), in Microns | | |
|---|---|---|---|
| | Curcumin | Lutein Zeaxanthin | LCD Oil |
| | Extract | Extract | Suspension |
| Before | 18.3 ± 1.9 | 17.5 ± 1.4 | 30.3 ± 2.5 |
| micronization | | | |
| After | 3.71 ± 0.4 | 2.12 ± 0.2 | 3.9 ± 0.3 |
| micronization | | | |

Particle Size Determination of a Test Product in Form of Oil Suspension

| | | | |
|---|---|---|---|
| Particle NameDry | Eye IAT Oil | Concentration | 0.0122% |
| Particle Refractive | Index 1.500 | Span | 3.002 |
| Particle Absorption | Index 0.010 | Uniformity | 0.929 |
| Dispersant Name | Liquid Paraffin | Specific Surface Area | 6380 m²/kg |
| Dispersant Refractive Index | 1.468 | D [3, 2] 0.940 μm ; | D [4, 3] 1.74 μm |
| Scattering Model | Mie | Dv (10) 0.467 μm ; | Dv (50) 1.14 μm |
| Analysis Model | General | Dv (90) 3.88 μm | |
| | Purpose | | |
| Weighted Residual | 6.62 % | | |

According to further embodiment of the present invention, the method for prevention, improvement and maintenance of dry eye syndrome, comprising a nutritional composition; wherein the subject is evaluated on oxidative stress markers and inflammatory markers related to dry eye syndrome.

According to further embodiment of the present invention the method for prevention, improvement and maintenance of dry eye syndrome, wherein a nutritional composition reduces inflammatory cytokines such as nuclear factor kappa light chain enhancer of activated B cells (NFkB), Matrix metallopeptidase 9 (MMP-9), tumor necrosis factor alpha (TNF-α), Interleukin 1 beta (IL-1b), Interleukin 6 (IL-6) and Interleukin 8 (IL-8).

According to further embodiment of the present invention the method for prevention, improvement and maintenance of dry eye syndrome, wherein a nutritional composition reduced oxidative stress marker such as malondialdehyde (MDA), superoxide dismutase (SOD), and glutathione peroxidase (GPx).

According to further embodiment of the present invention the method for prevention, improvement and maintenance of dry eye syndrome, wherein a nutritional composition increases tear volume, tear break-up time, tear stability, increased mucin production.

According to further embodiment of the present invention the method for prevention, improvement and maintenance of dry eye syndrome,—Corneal surface damage, Conjunctival surface damage, Artificial Tear Usage and Frequency.

In some embodiments, a process for the preparation of a nutritional composition comprises:
(i) Add one or more carrier with continue stirring, heat the solution at 65-95° C.
(ii) Add antioxidant into solution obtained by step (i) and homogenize till it completely dissolve. Maintain temperature of 65-95° C. for 20-30 min then allow the suspension to cool down below 30-40° C. for further process.
(iii) Add curcumin extract into solution obtained by step (ii) under stirring and maintain temperature in range 65-95° C.
(iv) Add bioavailability enhancing agent into solution obtained by step (iii)
(v) Add marigold extract and vitamin D3 oil to step (iv) dispersion, stir at 1200-2000 RPM for 20-30 min.
(vi) Add one or more excipient with continue stirring at 1200-2000 RPM
(vii) Sieving step 4 dispersion through mesh

At the outset of the description that follows, it is to be understood that the ensuing description only illustrates a particular form of this invention. However, such a particular form is only an exemplary embodiment and is not intended to be taken restrictively to imply any limitation on the scope of the present invention.

### Example 01

According to preferred embodiments we have performed different size batches from small to large scale to support the industrial viability in process wherein one batch of higher side as provided below as an example but no limited to batch size. The excipients ratio with respect to composition may be varied from the batch size so it should not limit the scope of invention. Oil suspension composition preparation; Batch size 9000 g.

| | Standard Quantity | |
|---|---|---|
| Ingredient | (g) | % w/w |
| Turmeric Extract (Curcumin) | 3031 | 33.68 |
| Marigold Extract (Lutein and | 485.5 | 5.39 |
| Zeaxanthin in ratio 4:1 to 6:1) | | |
| Vitamin D3 Oil | 9.63 | 0.107 |
| Linseed Oil | 900 | 10 |
| Mixed Tocopherol | 180 | 2 |
| d-limonene | 180 | 2 |
| Olive Oil | 450 | 5 |
| Lecithin | 180 | 2 |
| Thyme Oil | 450 | 5 |
| MCT(Medium Chain | 3133.87 | 34.821 |
| Triglyceride) Oil | | |
| Total | 9000 | 100 |

Process Details

Phase 1

i) Weigh all the ingredient wherein lutein: zeaxanthin is present in a ratio of 4:1 to 6:1 and

ii) Keep aside 10% of batch quantity of MCT oil for phase 2

iii) Add 90% of the batch quantity of MCT oil to the vessel/reactor and heat up to 65-95° C.

iv) Add batch quantity of lecithin in solution obtained by step (ii) and homogenize at 1000-3000 RPM for 20-30 min or till lecithin is completely dissolved. Maintain temperature of 65-95° C. for 20-30 min then allow the suspension to cool down below 30-40° C. for further process.

v) Add batch quantity of curcumin extract and homogenize at 1000-3000 RPM for 20-30 min.

vi) Add batch quantity of d-limonene, mixed tocopherol 700%-SF, thyme oil, olive oil, linseed oil to the vessel/reactor.

vii) Add batch quantity of marigold extract and homogenize at 1000-3000 RPM for 25-40 min or till homogenous suspension obtained.

Phase 2

i) Add batch quantity of vitamin D3 (Oil) to the vessel.

ii) Add Approx. 50% portion of remaining 10% quantity of MCT Oil to the vessel/reactor for mixing of vitamin D3(Oil) at 300-700 RPM for 2-5 min and use remaining quantity of MCT Oil for rinsing during transfer to the phase 3 suspension preparation vessel/reactor

Phase 3

i) Add phase 2 suspension into phase 1 suspension vessel/reactor and homogenize at 1000-3000 RPM for 3-5 min.

ii) Sieve through 20 #ASTM mesh followed by packaging.

Clinical Study:

Objective: To evaluate the efficacy of nutritional composition on subjects with Dry Eye Syndrome (DES)

Test Product (Actives): A Nutritional composition Capsule (Lutein/Zeaxanthin—20/4 mg+Curcumin—200 mg+Vitamin D3—600 IU) manufactured by OmniActive Health Technologies Limited, India.

Placebo: Soybean oil Capsule manufactured by OmniActive Health Technologies Limited, India

Study Design: A prospective, randomized, double-blind, multiple dose, parallel, placebo-controlled, clinical interventional study.

Duration of treatment: Total study duration for the clinical part was a maximum of 67 days which included the screening period of 8 days followed by the treatment period of 56 (8 weeks) followed by an End of study visit at 56±3 days.

Study Visits: Visit 1: Screening/Baseline Visit (Day −7 to Day 0), Visit 2: Randomization Visit (Day 0), Visit 3: First Follow-Up Visit (Day 14±3 days), Visit 4: Second Follow-Up Visit (Day 28±3 days), Visit 5: End of Treatment Visit (Day 56±3 days)

Dose and mode of administration: Subjects were instructed to consume one capsule every morning after breakfast, at the same time every day, for 56 days (8 weeks).

Number of volunteers: Approximately, 60 adult subjects aged between 18 and 65 with a clinical diagnosed Dry Eye Syndrome (DES) was planned for randomization. About 30 Subjects completed and analysed from Active Group and 29 subjects completed and analysed from the Placebo Group.

Primary Endpoints:

Tear volume; Ocular Surface Disease Index Score (OSDI)

Secondary Endpoints:

Tear film break-up time (TBUT); Standard Patient Evaluation of Eye Dryness (SPEED) score; tear osmolarity; corneal and conjunctival staining; MMP-9; artificial tear use

Schirmer's Test

Schirmer's test was used to measure aqueous tear production in the eyes. The strips were placed into the lower temporal lid margin of the eye for five minutes. The length of the wetness was measured in mm and interpreted for the severity of the dry eye syndrome. In this study, Schirmer's Test was evaluated at Baseline, Day 14, Day 28, and Day 56 of the investigational Test Product (Active) consumption.

TABLE 1
Summary of Mean Schirmer’s Test Strip Wetness Length in mm (Overall
Subjects-Average of both eyes) Between Test Product (Active) and
Placebo Groups.

| | | Placebo | P-value by ANOVA |
|---|---|---|---|
| | Active (N = 30) | (N = 29) | for Test product |
| Visit | Mean (SD) | Mean (SD) | (Active) vs. Placebo |
| Baseline | 6.62 (1.73) | 7.09 (2.45) | 0.3979 |
| Day 14 | 9.90 (4.21) | 8.14 (2.55) | 0.058 |
| Day 28 | 12.23 (3.18) | 8.69 (2.90) | <.0001* |
| Day 56 | 14.32(2.51) | 7.43 (3.29) | <.0001* |

N-number of subjects in specified treatment,
SD-Standard Deviation,
*P-value < 0.05

In between-group analysis using ANOVA test, the Active group showed a statistically significant increase in Mean Schirmer's Test strip wetness length (tear volume) over Placebo group on Day 28 and 56.

Ocular Surface Disease Index (OSDI) Scores

OSDI is a symptom-based questionnaire used for diagnosis of Dry Eye Syndrome. It is a twelve-item scale to assess the ocular surface symptoms of the dry eye, scores ranging from 0 to 100.

TABLE 2
Summary of Mean OSDI Score (Overall Subjects) Between Test Product
(Active) and Placebo Groups

| | Active | Placebo | P-value by ANOVA |
|---|---|---|---|
| | (N = 30) | (N = 29) | for Test product |
| Visit | Mean (SD) | Mean (SD) | (Active) vs. Placebo |
| Baseline | 30.10(4.88) | 32.54 (5.06) | 0.0642 |
| Day 14 | 25.24 (4.74) | 30.68 (5.48) | 0.0001* |
| Day 28 | 19.16(4.40) | 30.60 (5.91) | <.0001* |
| Day 56 | 17.09 (4.85) | 31.00 (7.18) | <.0001* |

N-number of subjects in specified treatment,
SD-Standard Deviation,
*P-value < 0.05

In between-group analysis using ANOVA test, the Active group showed a statistically significant decrease in Mean OSDI score over Placebo group at day 14, 28, and 56

Tear Break-Up Time (TBUT)

TBUT is defined as the time interval (in seconds) between a complete blink and the first appearance of a dry spot in the tear film after fluorescein administration. TBUT is used for assessing the stability of the tear film.

TABLE 3
Summary of Mean TBUT Score in seconds (Overall Subjects-Average of
Both Eyes) Between Test Product (Active) and Placebo Groups

| | Active | Placebo | P-value by ANOVA |
|---|---|---|---|
| | (N = 30) | (N = 29) | for Test product |
| Visit | Mean (SD) | Mean (SD) | (Active) vs. Placebo |
| Baseline | 8.18 (1.18) | 7.86 (1.25) | 0.3129 |
| Day 14 | 9.97 (1.80) | 7.97 (1.48) | <.0001* |
| Day 28 | 11.03 (1.32) | 7.93 (1.18) | <.0001* |
| Day 56 | 12.13 (1.38) | 6.86 (1.51) | <.0001* |

N-number of subjects in specified treatment,
SD-Standard Deviation,
*P-value < 0.05
In between-group analysis using ANOVA test, the Active group showed a statistically significant increase in Mean TBUT Score over Placebo group on Days 14, 28, and 56.

Tear Osmolarity (Tear Stability)

Tear film stability is diagnosed in dry eye syndrome subjects by measuring tear osmolarity. Tear osmolarity is measured in mOsms/L. A normal eye has less than 290 mOsms/L or 290-310 mOsmol/L with the variance between right and left eye ≤7 mOsmol/L.

TABLE 4
Summary of Mean Tear Osmolarity (Overall Subjects-Average of Both
Eyes) Between Test Product (Active) and Placebo Groups

| | Active | Placebo | P-value by ANOVA |
|---|---|---|---|
| | (N = 30) | (N = 29) | for Test product |
| Visit | Mean (SD) | Mean (SD) | (Active) vs. Placebo |
| Baseline | 327.23 (12.42) | 327.34(11.06) | 0.9711 |
| Day 56 | 320.42 (12.24) | 331.72(11.20) | 0.0005* |

N-number of subjects in specified treatment,
SD-Standard Deviation,
*P-value < 0.05
In between group analysis using the ANOVA test, the Active group showed a statistically significant increase (p < 0.05) in tear osmolarity over the Placebo group on Day 56

Standardized Patient Evaluation of Eye Dryness (SPEED) Questionnaire

SPEED is an 8-items questionnaire to assess the frequency and severity of dry eye symptoms.

The questionnaire contains scores from 0 to 28. Additionally, the questionnaire analyses the symptoms further into its severity from non-problematic to intolerable.

TABLE 5
Summary of Mean SPEED Score (Overall Subjects) Between Test Product
(Active) and Placebo Groups

| | Active | Placebo | P-value by ANOVA |
|---|---|---|---|
| | (N = 30) | (N = 29) | for Test product |
| Visit | Mean (SD) | Mean (SD) | (Active) vs. Placebo |
| Baseline | 11.63 (1.25) | 12.21 (1.78) | 0.1559 |
| Day 14 | 8.97 (1.30) | 11.03 (2.23) | <.0001* |
| Day 28 | 7.07 (1.36) | 10.31 (2.16) | <.0001* |
| Day 56 | 6.07 (1.86) | 12.41 (2.90) | <.0001* |

N-number of subjects in specified treatment,
SD-Standard Deviation,
*P-value < 0.05
In between-group analysis using ANOVA test, the Active group showed a statistically significant decrease in Mean SPEED Score over Placebo group at day 14, 28, and 56.

Matrix metalloproteinase-9 (MMP-9) Biomarker for Tear Inflammation

Matrix metalloproteinase-9 (MMP-9) is a biomarker released in tear due to inflammatory response in dry eye syndrome subjects. The presence of MMP-9 was tested at baseline and the end of the study (Day 56). Presence of MMP-9 level <40 ng/ml is normal and considered as negative.

TABLE 6
Number of Subjects Positive for MMP-9 Biomarker (Overall Subjects-
Right Eye) Between Test Product (Active) and Placebo Groups

| | | | | | P-value by Z Test |
|---|---|---|---|---|---|
| | Active (N = 30) | | Placebo (N = 29) | | for Test product |
| | Negative | Positive | Negative | Positive | (active) vs |
| Visit | n (%) | n (%) | n (%) | n (%) | Placebo |
| Baseline | 14 (46.67) | 16 (53.33) | 14 (48.28) | 15 (51.72) | 0.9015 |
| Day 56 | 28 (93.33) | 2 (6.67) | 17 (58.62) | 12(41.38) | 0.0017* |

N-Number of subjects in specified treatment;
n-number of subjects in specified category.
% = n (number of subjects in specified category)/N (number of subjects in specified treatment) × 100.
*P-value < 0.05

In between-group analysis using Z-test, Test product (active) group showed a significant reduction in the number of subjects positive for Tear Inflammation as measured by MMP-9 marker on Day 56 compared to the Placebo group.

TABLE 7
Number of Subjects Positive for MMP-9 Biomarker (Overall Subjects-
Left Eye) Between Test product (Active) and Placebo Groups

| | | | | | P-value by Z |
|---|---|---|---|---|---|
| | | | | | Test for Test |
| | Active (N = 30) | | Placebo (N = 29) | | product |
| | Negative | Positive | Negative | Positive | (active) vs |
| Visit | n (%) | n (%) | n (%) | n(%) | Placebo |
| Baseline | 14 (46.67) | 16 (53.33) | 15 (51.72) | 14 (48.28) | 0.6977 |
| Day 56 | 28 (93.33) | 2 (6.67) | 17 (58.62) | 12(41.38) | 0.0017* |

N-Number of subjects in specified treatment;
n-number of subjects in specified category.
% = n (number of subjects in specified category)/N (number of subjects in specified treatment) × 100.
*P-value < 0.05

In between group analysis using the Z-test, Test product (active) group showed a significant reduction in the number of subjects positive for Tear Inflammation as measured by MMP-9 marker on Day 56 compared to the Placebo group.

Artificial Tear Usage and Frequency

Artificial Tears are used for relieving the symptoms of dry eyes by providing lubrication, decreasing tear osmolarity, and increasing viscosity. Usage of artificial tears is palliative. During the study, subjects were prescribed hydroxypropyl methylcellulose 0.7% w/v at the randomization visit, from Day 14 (visit 3) till End of Study (Day 56).

TABLE 8
Summary of Number of Subjects Used Artificial Tears (Overall Subjects)
Between Test Product (Active) and Placebo Groups

| | | | | | P-value by Z |
|---|---|---|---|---|---|
| | | | | | Test for Test |
| | Active (N = 30) | | Placebo (N = 29) | | product |
| | No | Yes | No | Yes | (active) vs |
| Visit | n (%) | n (%) | n (%) | n (%) | Placebo |
| Day 0-14 | 15 (50.00) | 15 (50.00) | 9 (31.03) | 20 (68.97) | 0.1382 |
| Day 15-28 | 23 (76.67) | 7 (23.33) | 9 (31.03) | 20 (68.97) | 0.0004* |
| Day 29-56 | 23 (76.67) | 7 (23.33) | 9 (31.03) | 20 (68.97) | 0.0004* |

N-Number of subjects in specified treatment;n-number of subjects in specified category.
% = n (number of subjects in specified category)/N (number of subjects in specified treatment) × 100.
*P-value < 0.05

Table 9: Summary from day 0 to day 14, 15 (50%) subjects used artificial tears in the Active group, and 20 (68.97%) subjects in the Placebo group.

From Day 15 onwards till Day 56, 7 (23.33%) subjects in the Active group and 20 (68.97%) subjects in the Placebo group continued using artificial tears. There was a percentage change of −53.33% from Baseline (Day 0 to Day 14) to Day 28 and remained same till Day 56 for Active group.

The Active group showed a significant reduction in the number of subjects using Artificial Tears from Day 15-28 and Day 29-56 compared to the Placebo group.

TABLE 9
Summary of Mean Frequency of Artificial Tears Usage (Overall Subjects)
Between Active and Placebo Groups

| | Active | Placebo | P-value by Kruskal- |
|---|---|---|---|
| | (N = 15) | (N=20) | Wallis test for Active |
| Visit | Mean (SD) | Mean (SD) | Vs Placebo |
| Day 0-14 | 2.47 (100.0) | 2.65 (100.0) | 0.2851 |
| Day 15-28 | 1.07 (43.24) | 2.45 (92.45 ) | 0.0009* |
| Day 29-56 | 0.73 (29.73 ) | 2.60 (98.11 ) | <.0001* |

N-Number of subjects in specified treatment;
*P-value <0.05

From Day 0 to Day 14, the artificial tear was used 2.47 times/day (100%) in the Active group and 2.65 times/day (100%) in the Placebo group. A gradual reduction in the frequency of use was found in the Active group from Day 15-28 and Day 29-56 respectively where it was used only 1.07 times/day (43.24%) i.e. 64.4±40.76% reduction from Baseline and 0.73 times/day (29.73%) i.e. 75.6±34.43% reduction from Baseline. In the Placebo group, artificial tears were used for 2.45 times/day (92.45%) and 2.60 times/day (98.11%) respectively from Day 15-28 and 29-56 (Table 30) with 9.17±19.10% and 0.83±32.21% reduction from Baseline.

In between-group analysis using Kruskal-Wallis test, the Active group showed a significant reduction in the frequency of Artificial Tears usage from Day 15-28 and Day 29-56 compared to the Placebo group.

Corneal Staining Scores

Fluorescein Stain is used to examine the condition of the ocular surface damage, i.e. corneal and conjunctival surface damages, in dry eye syndrome subjects. Fluorescein is a synthetic organic compound which is an orange-red colored quick-acting temporary dye. Fluorescein stains the damages on the ocular surface as bright green under blue light (slit lamp). Each eye is scored separately based on the damages observed by the study investigator.

TABLE 10
Summary of Mean Corneal Staining Score (Overall Subjects-Average of
Both eyes) Between Test Product (Active) and Placebo Groups

| | Active | Placebo | |
|---|---|---|---|
| | (N = 30) | (N = 29) | |
| Visit | Mean (SD) | Mean (SD) | Active vs Placebo |
| Baseline | 1.14 (0.17) | 1.10 (0.17) | 0.3965 |
| Day 56 | 0.79 (0.25) | 1.24 (0.20) | <.0001* |

N-Number of subjects in specified treatment.
SD-Standard Deviation.
*P-value < 0.05

Conjunctival Staining Scores

The same method as corneal staining is followed for conjunctival staining score.

TABLE 11
Summary of Mean Conjunctival Staining Score (Overall Subjects-Average
of Both eyes) Between Test Product (Active) and Placebo Groups

| | Active | Placebo | |
|---|---|---|---|
| | (N = 30) | (N = 29) | P-value by ANOVA for |
| Visit | Mean (SD) | Mean (SD) | Active vs Placebo |
| Baseline | 0.88 (0.22) | 0.86 (0.20) | 0.7775 |
| Day 56 | 0.58 (0.32) | 1.03 (0.27) | <.0001* |

N-Number of subjects in specified treatment.
SD-Standard Deviation.
*P-value < 0.05

Results from Pre-Clinical Study Using Dry Eye Animal Model

Seven female Wistar rats per treatment arm (age: 8 weeks, weight: 180±20 g) were housed in a controlled environment with a 12:12-h light-dark cycle at 22° C. and were provided with rat chow and water ad libitum.

Dry eye disease was established by the topical administration of benzalkonium chloride (BAC) solution (0.2%, Sigma-Aldrich,) to rat eyes twice daily for 14 days. The rats were divided randomly to 4 groups:
I. normal control group (n=7),
II. vehicle control group (n=7),
III. blend formulation group 1—LCD I (100 mg/kg bw) treated group (n=7) and,
IV. blend formulation group 2—LCD II (200 mg/kg bw) treated group (n=7)

Blend formulation was given by oral gavage for 4 weeks.

TABLE 11
Effect of the combination of lutein, zeaxanthin, curcumin and vitamin D (LCD) on
serum malondialdehyde (MDA), corneal MDA, corneal superoxide dismutase (SOD), and
corneal glutathione peroxidase (GSH-Px), in benzalkonium chloride (BAC) induced dry eye
syndrome in rats.

| | Mean ± SD | | | |
|---|---|---|---|---|
| | | Corneal | | |
| Groups | MDA Serum | MDA | Corneal SOD | Corneal GPx |
| Normal Control | 0.534 ± 0.03 | 0.42 ± 0.05 | 19.10 ± 1.50 | 72.50 ± 8.47 |
| Vehicle Control | 0.794 ± | 1.87 ± 0.29**** | 9.84 ± 1.21**** | 44.86 ± 6.49**** |
| | 0.05**** | | | |
| LCD I | 0.689 ± | 1.25 ± | 12.64 ± | 53.71 ± 5.96*** |
| | 0 03****^{, ###} | 0.43****^{, ##} | 1 16****^{, ##} | |
| LCD II | 0.619 ± | 1.06 ± | 15.26 ± | 61.14 + 7.58*^{, ##} |
| | 0 04**^{, ####, $} | 0.13***^{, ####} | 0.92****^{, ####, $$} | |

ANOVA and Turkey's post-hoc test. Statistical significance between groups is shown by:
*P < 0.05;
**P < 0.01;
***P < 0.001;
****P < 0.0001 compared as control group;
^{#}P < 0.05;
^{##}P < 0.01;
^{###}P < 0.001;
^{####}P < 0.0001 compared as Dry Eye group;
^{$}P < 0.05;
^{$$}P < 0.01;
^{$$$}P < 0.001;
^{$$$$}P < 0.0001 compared as Dry Eye + LCD I group).

Normal control Group—group of normal rats wherein no test product or vehicle was given to dry eye syndrome rat group.

Vehicle control Group—BAC-induced dry eye syndrome vehicle was administered.

LCD I Group—group of rats wherein 100 mg/kg body weight of test product (Lutein, zeaxanthin, curcumin and Vitamin D3) was given to dry eye syndrome rat group.

LCD II Group—group of rats wherein 200 mg/kg body weight of test product (Lutein, zeaxanthin, curcumin and Vitamin D3) was given to dry eye syndrome rat group.

The data indicates that LCD formulation at both doses significantly reduced oxidative stress initiated by BAC-induced DES by restoring the levels of MDA and improving the levels of antioxidants SOD and GSH-Px The effects of BAC and LCD on corneal enzyme activities of SOD and GSH-Px and MDA levels in serum and the cornea. BAC induced oxidative stress in the ocular surface and increased serum & corneal levels of MDA, decreased corneal levels of SOD and GSH-Px, whereas LCD partially ameliorated the effect of oxidative stress induced by BAC (p<0.05). The improvement was more obvious in the LCD 2 group compared with the LCD 1 group (p<0.05).

Histological Analyses

Eyes were fixed (4% paraformaldehyde and then paraffin) and sectioned into 5-μm slices using a microtome. Corneal and conjunctival tissues were stained with hematoxylin and eosin (H&E) and examined using light microscopy.

TABLE 12

| | Mean ± SD | |
|---|---|---|
| | Corneal epithelial | |
| Groups | thickness, μm | Corneal thickness, μm |
| Normal Control | 54.45 ± 4.73 | 149.66 ± 8.32 |
| Vehicle Control | 28.89 ± 2.14**** | 89.56 ± 5.68**** |
| LCD I | 35.09 ± 1.88****^{, #} | 108.23 ± 10.51****^{, #} |
| LCD II | 41.84 ± 3,05***^{, ####, $} | 128.53 ± 9,87**^{, ####, $} |

ANOVA and Turkey's post-hoc test. Statistical significance between groups is shown by:
*P < 0.05;
**P < 0.01;
***P < 0.001;
****P < 0.0001 compared as control group;
#P < 0.05;
^{##}P < 0.01;
^{###}P < 0.001;
^{####}P < 0.0001 compared as Dry Eye group;
^{$}P < 0.05;
^{$$}P < 0.01;
^{$$$}P < 0.001;
^{$$$$}P < 0.0001 compared as Dry Eye + LCD I group).

We observed that BAC-induced dry eye condition resulted in increased cormeal epithelial thickness and inflammatory cell infiltration and edema under the epithelium were observed. LCD caused an improvement in the histopathologic findings in a dose-dependent fashion.

Western Blot Analyses

Muc1, Muc4, Muc5, GAP43, GFAP, NF-κB, TNF-α, IL-1β, IL-6, and IL-8 was measured in corneal tissues using Western Blot analysis. Monoclonal mouse antibody against β-actin (A5316; Sigma) was used as the loading control. Blots were performed at least three times to confirm the reproducibility of the results. The densitometric analyses of bands were detected with an image analysis system, Image J (National Institute of Health, Bethesda, Md., USA).

TABLE 13

| | Mean ± SD | | | | | | | |
|---|---|---|---|---|---|---|---|---|
| Groups | NF-kB | TNF-α | IL-1β | IL-6 | IL-8 | Mucin 1 | Mucin 4 | Mucin 5 |
| Normal | 99.999 ± | 100.000 ± | 100.000 ± | 100.000 ± | 100.000 ± | 100.000 ± | 99.999 ± | 99.997 ± |
| Control | 12.95 | 3.16 | 16.77 | 12.38 | 9.15 | 11.40 | 4.40 | 10.84 |
| Vehicle | 142.479 ± | 146.429 ± | 206.199 ± | 184.710 ± | 189.703 ± | 57.997 ± | 36.396 ± | 39.234 ± |
| Control | 15.60**** | 8.55**** | 11.68**** | 8.97**** | 10.10**** | 11.42**** | 13.26**** | 7.28**** |
| LCD I | 114.787 ± | 131.231 ± | 146.750 ± | 159.263 ± | 156.377 ± | 75.219 ± | 58.231 ± | 59.950 ± |
| | 10.37^{##} | 9.4****^{,##} | 7.39****^{,####} | 8.29****^{,###} | 5.27****^{,####} | 5.06***^{,#} | 7.86****^{,##} | 9.59****^{,###} |
| LCD II | 92.280 ± | 119.227 ± | 112.247 ± | 116.451 ± | 126.907 ± | 82.616 ± | 62.927 ± | 66.390 ± |
| | 16.85^{####,$} | 4.66***^{,####,$} | 6.11^{####,$$$$} | 6.37*^{,####,$$$$} | 4.64****^{,####,$$$$} | 7.87**^{,###} | 11.90****^{,###} | 2.54****^{,####} |

ANOVA and Turkey’s post-hoc test. Statistical significance between groups is shown by:
*P < 0.05;
**P < 0.01;
***P < 0.001;
****P < 0.0001 compared as control group;
^{#}P < 0.05;
^{##}P < 0.01;
^{###}P < 0.001;
^{####}P < 0.0001 compared as Dry Eye group;
^{$}P < 0.05;
^{$$}P < 0.01;
^{$$$}P < 0.001;
^{$$$$}P < 0.0001 compared as Dry Eye + LCD 1 group.

BAC-induced eye symptoms were associated with significant increase in levels of NF-κB, TNF-α, IL-1β, IL-6, IL-8 and decrease in Muc1, Muc4, Muc5, GAP43 proteins (p<0.001). However, LCD reversed the above protein expressions with LCD2 being more efficient than LCD1 (p<0.05).

1) A composition comprising:
a) an extract comprising lutein and zeaxanthin;
b) curcumin;
c) vitamin D3; and
d) at least one excipient.

2) The composition according to claim 1, wherein the at least one excipient is selected from the group consisting of an antioxidant, a bioavailability enhancing agent, a carrier, a fat, a solubilizer, a stabilizer, and a combination thereof.

3) The composition according to claim 2, wherein the antioxidant is selected from the group consisting of tocopherols, ascorbyl palmitate, rosemary extract, epigallocatechin gallate, catechins, ascorbic acid, and a combination thereof.

4) The composition according to claim 2, wherein the bioavailability enhancing agent is selected from the group consisting of olive oil, thyme oil, linseed oil, d-limonene, lecithin, monoglycerides, phosphatidyl choline, a botanical extract, and a combination thereof.

5) The composition according to claim 2, wherein the carrier is selected from the group consisting of milk fat, medium chain triglycerides (MCT), long chain triglycerides, flaxseed oil, olive oil, thyme oil, fish oil, algal DHA oil, krill oil, safflower oil, sunflower oil, soybean oil, coconut oil, vegetable oil, esters of fatty acids, hydrocarbons, terpenes, monoglycerides, oils, and a combination thereof.

6) The composition according to claim 1, wherein the extract is present in a particle size ranging from 0.1 micron to 10 micron.

7) The composition according to claim 1, wherein the curcumin is present in a particle size ranging from 0.1 micron to 10 micron.

8) The composition according to claim 1, wherein the composition is in a form selected from the group consisting of beadlets, a powder, a form oil suspension, granules, capsules, tablets, and films.

9) A method for treating, preventing, and/or improving dry eye syndrome in a subject, comprising administering the composition according to claim 1 to a subject in need thereof.

10) The according to claim 9, wherein the administering of the composition reduces concentration of one or more inflammatory cytokines selected from the group consisting of nuclear factor kappa light chain enhancer of activated B cells (NFkB), matrix metallopeptidase 9 (MMP-9), tumour necrosis factor alpha (TNF-a), Interleukin 1 beta (IL-1b), Interleukin 6 (IL-6), Interleukin 8 (IL-8), and a combination thereof, in the subject.

11) The method according to claim 9, wherein the administering of the composition reduces concentration of at least one oxidative stress marker in the subject.

12) The method according to claim 9, wherein the administering of the composition results in one or more beneficial effects selected from the group consisting of increased tear volume, increased tear break-up time, increased tear stability, increased mucin production, reduced corneal surface damage, reduced conjunctival surface damage, reduced artificial tear usage and frequency, enhanced tear production, soothing dry eyes, reduced eye discomfort, improved tear stability, reduced irritation, burning and grittiness associated with dry eyes, reduced tear loss, reduced ocular surface damage, reduced inflammation in tears, reduced dependence on artificial tears, and a combination thereof, in the subject.

13) (canceled)

14) (canceled)

15) The method according to claim 9, wherein the subject is a human.

16) The method according to claim 9, wherein the composition is administered in an amount ranging from 10 mg/kg to 500 mg/kg.

17) The method according to claim 16, wherein the composition is administered orally.

18) The method according to claim 16, wherein the composition is administered once a day.

19) The method of claim 9, wherein the administration of the composition increases concentration of one or more antioxidant enzymes in the subject.

20) The method of claim 19, wherein the one or more antioxidant enzymes is selected from the group consisting of superoxide dismutase (SOD), glutathione peroxidase (GPx), and a combination thereof.

21) The method according to claim 9, wherein the administering of the composition reduces concentration of at least one oxidative stress marker in the subject; wherein the at least one oxidative stress marker is malondialdehyde (MDA).

22) A composition comprising:
a) an extract comprising lutein and zeaxanthin;
b) curcumin; and
c) vitamin D3.

23) A process for preparing a composition according to claim 1, wherein the composition is in the form of an oil suspension, the process comprising:
a) preparing solution of one or more carrier and bioavailability enhancing agent;
b) adding curcumin extract to the solution followed by an antioxidant and the extract comprising lutein and zeaxanthin;
c) preparing separate solution of Vitamin D3 and carrier;
d) homogenizing the mixture obtained by adding step (b) and (c); and
e) sieving step (d) dispersion through a mesh.
